(19) United States
(12) Patent Application Publication
(10) Pub. No.: US 2026/0249356 A1
(43) Pub. Date: Aug. 27, 2026

(54) ADDITIVELY MANUFACTURED WIRE PRODUCTS FORMED FROM NICKEL-TITANIUM SHAPE MEMORY ALLOYS

(71) Applicant: Fort Wayne Metals Research Products, LLC, Fort Wayne, IN (US)

(72) Inventors: Kyle Fezi, Fort Wayne, IN (US); Adam J. Griebel, Hoagland, IN (US); Drew Forbes, Fort Wayne, IN (US); Jeremy E. Schaffer, Fort Wayne, IN (US)

(21) Appl. No.: 19/534,157

(22) Filed: Feb. 9, 2026

Related U.S. Application Data
(60) Provisional application No. 63/756,475, filed on Feb. 10, 2025.

(51) Int. Cl. B22F 10/28; B22F 10/64; B22F 10/66; B33Y 10/00; B33Y 40/20; B33Y 80/00

(52) U.S. Cl. CPC B22F 10/28; B22F 10/64; B22F 10/66; B33Y 10/00; B33Y 40/20; B33Y 80/00; B22F 2301/15; B22F 2304/10; B22F 2998/10; B22F 2999/00

(57) ABSTRACT

A method of producing a wire product from a nickel-titanium shape memory alloy, and a wire product produced by such method. The method includes consolidating a nickel-titanium shape memory alloy to a preform using an additive manufacturing process. Thereafter, the preform may be subject to a hot working process, including heating the preform and deforming the preform, such as by, forging, extrusion, rolling, swaging, and the like. The hot-worked preform may be annealed following hot working. The preform may then be subjected to one or more cold working processes including drawing, swaging, rolling, and the like. Annealing may also occur after cold working. The hot and cold working processes may impart significant diameter reduction to form the wire product.

### CROSS REFERENCE TO RELATED APPLICATIONS

This application claims the benefit of U.S. Provisional Application No. 63/756,475 filed Feb. 10, 2025, entitled “ADDITIVELY MANUFACTURED WIRE PRODUCTS FORMED FROM NICKEL-TITANIUM SHAPE MEMORY ALLOYS” which is herein incorporated by reference in its entirety.

### BACKGROUND

### 1. Technical Field

The present disclosure is directed to a wire and/or wire construct for use in medical device and/or applications and formed of a nickel-titanium (NiTi) shape memory alloy, such as Nitinol, via additive manufacturing in combination with iterative deformation and optional annealing steps, the wire product having high microstructural cleanliness.

### 2. Description of the Related Art

Wire products for use in medical device applications such as electrical leads and stents may be formed of nickel-titanium shape memory alloys such as Nitinol, a binary shape memory alloy of nickel and titanium, and/or ternary and greater alloys thereof.

Wire products formed of nickel-titanium shape memory alloys inherently include non-metallic inclusions that are formed in the melt process and, in high fatigue applications, crack formation stemming from such inclusions is possible. Non-metallic inclusions in nickel-titanium shape memory alloys are generally rich in either carbon or oxygen, and the presence and form of such inclusions, often referred to as microcleanliness, is specified by ASTM F2063.

Various strategies are employed during the melt process of nickel-titanium shape memory alloys to reduce the number and size of inclusions. For example, high purity nickel and titanium raw materials and high purity vacuum environments can reduce total oxygen brought into the system during the melt process. Further, “gettering” elements such as yttrium may be intentionally included in the melt in minor amounts to favorably interact with oxygen present in the melt to mitigate formation of oxide-based inclusions. Other elements may also be deployed in the melt which refine grain structures of the cast material, including carbon, to aid in minimizing inclusion size. Still further, smaller ingot size, resulting in faster solidification, is also known to reduce as-cast grain size and overall inclusion size.

Wire products are formed from ingots formed via the melt process, typically by a large number of iterative deformation and annealing steps, which may include hot working steps, to achieve a large extent of diameter/cross sectional reduction. However, with conventional melt processes, the presence of inclusions in nickel-titanium shape memory alloys is inevitable, even when mitigation methods are used, and improvements in the microcleanliness of wire products formed of nickel-titanium shape memory alloys is desired.

### SUMMARY

The present disclosure is directed to a method of producing a wire product from a nickel-titanium shape memory alloy, and a wire product produced by such method. The method includes consolidating a nickel-titanium shape memory alloy to a preform using an additive manufacturing process. Thereafter, the preform may be subject to a hot working process, including heating the preform and deforming the preform, such as by forging, extrusion, rolling, swaging, and the like. The hot-worked preform may be annealed following hot working. The preform may then be subjected to one or more cold working processes including drawing, swaging, rolling, and the like. Annealing may also occur after cold working. The hot and cold working processes may impart significant diameter reduction to form the wire product.

Advantageously, the additive manufacturing of the preform, such as by laser sintering, wire arc additive manufacturing, or electron beam manufacturing, forms a consolidated preform having a very clean microstructure with minimized inclusions, which results in the wire product also having the desired microstructure. In particular, the resulting wire product may have at least 96%, 97%, or 98.5% of all total visible inclusions having a longitudinal maximum dimension of 5 microns (μm) or less.

The wire products of the present disclosure may be used in medical device applications, or in a variety of other applications in aerospace, automotive and/or industrial applications, such as shape memory actuators, for example.

In one form thereof, the present disclosure provides a method of producing a wire product from a nickel-titanium shape memory alloy, comprising consolidating a nickel-titanium shape memory alloy to a preform using an additive manufacturing process, the preform having a first cross-sectional area; and deforming the preform to manufacture a wire product having a second cross-sectional area, the second cross-sectional area being at least 30% less than the first cross-sectional area.

In another form thereof, the present disclosure provides a wire product formed of a nickel-titanium shape memory alloy comprising at least 96% of all total visible inclusions having a longitudinal maximum dimension of from 5 microns to 1 micron and a rotary beam fatigue strength exceeding 10,000 cycles at 1% strain.

In another form thereof, the present disclosure provides a method of producing a wire product from a nickel-titanium shape memory alloy, comprising consolidating a nickel-titanium shape memory alloy to a preform using an additive manufacturing process, the preform having a first cross-sectional area; heating the preform; deforming the heated preform to reduce the first cross-sectional area of the preform to a second cross-sectional area; annealing the deformed preform; and deforming the preform at least once to form a wire product having a third cross-sectional area, the deformation step achieving a cross-sectional area reduction, wherein the third cross sectional area is at least 30% less than the first cross-sectional area.

In another form thereof, the present disclosure provides a medical device formed from a wire product formed of a nickel-titanium shape memory alloy.

### BRIEF DESCRIPTION OF THE DRAWINGS

The above mentioned and other features and objects of this invention, and the manner of attaining them, will become more apparent and the invention itself will be better understood by reference to the following description of embodiments of the invention taken in conjunction with the accompanying drawings, wherein:

FIG. 1 is an illustration of a powder bed additive manufacturing system/apparatus.

FIG. 2 is a cross-sectional fragmentary view of a laser melting powder material.

FIG. 3 is an illustration of a wire-based additive manufacturing system/apparatus.

FIG. 4 is a cross-sectional view of melted wire material being used in an additive manufacturing process.

FIG. 5 is flow chart illustrating steps in the manufacturing process of the present disclosure.

FIG. 6 corresponds with Example 1 and is a histogram of the maximum length of nonmetallic inclusion void assemblies for traditionally manufactured NiTi #5

FIG. 7 corresponds with Example 1 and is a histogram of the maximum area percent of nonmetallic inclusion void assemblies for traditionally manufactured NiTi #5.

FIG. 8 corresponds to Example 1 and is a micrograph of a NiTi #5 0.275″ coil manufactured with traditional processes showing NMI area fraction of 3.26 with a 500× magnification.

FIG. 9 corresponds to Example 1 and is a micrograph of a NiTi #5 0.275″ coil manufactured with traditional processes showing and showing 68.6 μm NMI with a magnification of 1000×.

FIG. 10 corresponds to Example 1 and is a micrograph of NiTi #5 wire manufactured from an additively manufactured preform showing no NMIs taken from 0.130″ diameter wire at 80× magnification.

FIG. 11 corresponds to Example 1 and is a micrograph of NiTi #5 wire manufactured from an additively manufactured preform showing no NMIs taken from 0.085″ diameter wire at 1000× magnification.

FIG. 12. corresponds to Example 1 and is micrograph of 4.52 mm diameter EOS printed NiTi #5 at 500×, wire showing 0.0 μm nonmetallic inclusion void assembly maximum length and 0.00% maximum area, demonstrating microstructure cleanliness.

FIG. 13 corresponds to Example 1 and is a summary of 30 tensile tests of 0.001″ diameter Nitinol #5 wire from different Nitinol suppliers showing the improved strength using wrought material manufactured with additive manufacturing.

FIG. 14 corresponds to Example 1 and is a summary of 30 tensile tests of 0.001″ diameter Nitinol #5 wire from different Nitinol suppliers showing the improved elongation using wrought material manufactured with additive manufacturing.

FIGS. 15A and 15B correspond with Example 4 and are longitudinal views of conventionally produced nitinol wire input and additively manufactured and drawn wire.

FIGS. 16A and 16B are exemplary thermomechanical trainers used as an actuator cycling durability testing apparatus.

FIG. 17 is an exemplary braided construct formed from additively manufactured NiTi wire.

FIG. 18 is an exemplary wire-form stent formed from additively manufactured NiTi wire.

FIG. 19 is an exemplary coil assist stent formed from additively manufactured NiTi wire.

FIG. 20 is an exemplary septal occlusion device formed from additively manufactured NiTi wire.

FIG. 21 is an exemplary biostimulation device including a cable formed from additively manufactured NiTi wire.

Corresponding reference characters indicate corresponding parts throughout the several views. Although the exemplifications set out herein illustrate embodiments of the invention, the embodiments disclosed below are not intended to be exhaustive or to be construed as limiting the scope of the invention to the precise form disclosed.

### DETAILED DESCRIPTION

### 3. Introduction

The present disclosure is directed to a method of producing a wire product from a nickel-titanium (e.g., Nitinol/Nitinol alloy) shape memory alloy, and a wire product produced by such method. The method includes consolidating a nickel-titanium shape memory alloy to a preform using an additive manufacturing process. Thereafter, the preform is subjected to, optionally, hot working, and then multiple drawing and optional annealing steps to achieve a significant diameter reduction to form the wire product. Advantageously, the additive manufacturing of the preform results in a very clean microstructure with minimized inclusions, whereas the resulting wire product also has the desired microstructure.

### 4. Definitions

As used herein, “wire” or “wire product” encompasses continuous wire and wire products which may be continuously produced and wound onto a spool for later dispensation and use, such as wire having a round cross section and wire having a non-round cross section, including flat wire or ribbon. “Wire” or “wire product” also encompasses other wire-based products such as strands, cables, coil, and tubing, which may be produced at a particular length depending on a particular application. In some exemplary embodiments, a wire or wire product in accordance with the present disclosure may have a diameter up to 2.5 mm or 0.25 inches, or greater as desired. Thin material sheets may also be made. Exemplary tubing structures may be in wire form or rod form, with inside diameters ranging from 0.5 mm to 4.0 mm, and wall thicknesses ranging from 0.100 mm to 1.00 mm. “Fine wire” refers to a wire having an outer diameter of less than 1 mm.

As used herein, “fatigue strength” refers to the load level at which the material meets or exceeds a given number of load cycles to failure. Herein, the load level is given as alternating strain, as is standard for displacement or strain-controlled fatigue testing, whereby terms align with those given in ASTM E606, the entirety of which is incorporated herein by reference.

“DFT®” is a registered trademark of Fort Wayne Metals Research Products LLC of Fort Wayne, IN, and refers to a bimetal or poly-metal composite wire product including two or more concentric layers of metals or alloys, typically at least one outer layer disposed over a core filament formed by drawing a tube or multiple tube layers over a solid metallic wire core element.

“Nickel-titanium shape memory alloy” is an alloy comprising nickel and titanium as principal elements, which alloy exhibits superelastic, or shape memory, characteristics.

“Binary nickel-titanium shape memory alloy” refers to alloys comprising nickel and titanium as principal elements, along with any impurities that may be present.

“Ternary nickel-titanium shape memory alloy” refers to alloys comprising nickel and titanium as principal elements, along with a third element, together with any impurities that may be present.

“Nitinol” is a trade name for a shape memory alloy comprising approximately 50 atomic % nickel and balance Titanium, also known as NiTi, commonly used in the medical device industry for highly elastic metallic implants.

“Impurities,” “incidental impurities” and “trace impurities” are material constituents present in a material at less than 500 parts per million or 0.05 wt. % for any given element.

### 5. Additive Manufacturing Systems and Methods for Forming Wire Products

Additive manufacturing (AM; also referred to herein as 3D printing) is a process of building objects layer by layer directly from digital 3D models. Unlike traditional subtractive manufacturing, which removes material, AM adds material only where needed, minimizing waste. AM allows for numerous technological advantages, such as enabling the production of complex geometries, reducing waste by using only the material required for the object, and facilitating tailored designs for specific applications or individual requirements.

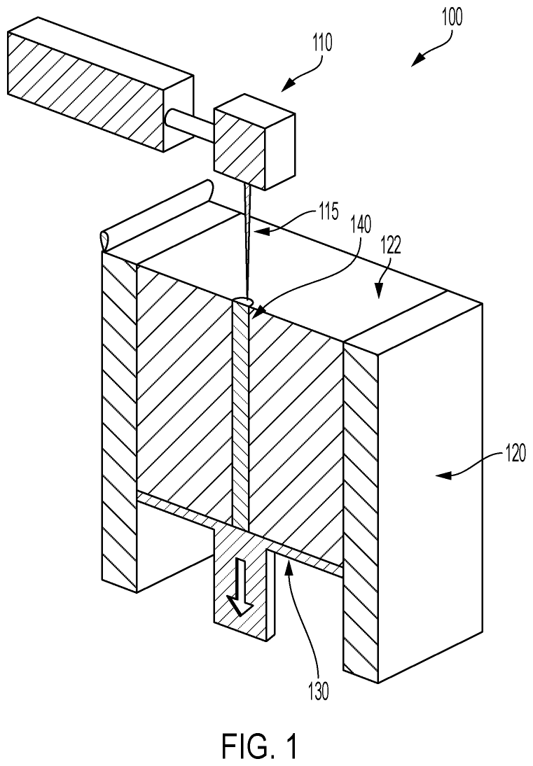

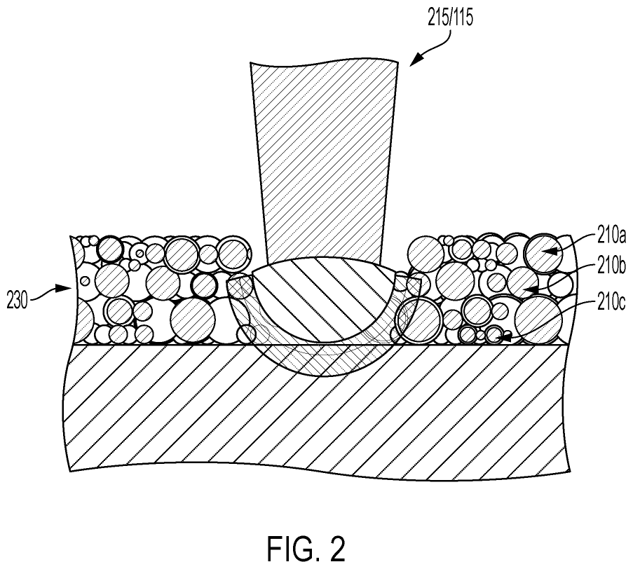

One type of AM technique is laser sintering. FIG. 1 illustrates a lateral side/cut view of an additive manufacturing system 100 utilizing laser sintering. System 100 includes laser system 110, powder bed 120, and build platform 130. Here, a powdered material 122, such as a metal powder of nitinol (e.g., to be described with further detail herein), is successively layered horizontally, in a direction perpendicular to build platform 130 within the sidewalls of powder bed 120. Laser beam 115, generated and emitted from laser system 110, contacts powder material 122 at a focal point, selectively sintering the powder material 122 in a predefined path, melting the powder material. FIG. 2, illustrates particularly, a laser beam 215, which may be the same as laser beam 115, melting particles of Nitinol 210 of various size (e.g., 210a, 210b, 210c) in a powder layer 205. Returning to FIG. 1, the melted powder material 122 solidifies/consolidates, and forms a layer of a three-dimensional object, such as preform 140. Once a layer of preform 140 has been sintered, the build platform 130 descends vertically (e.g., downward, in the Z direction), and a subsequent layer of powder material 122 is added to the top of powder bed 120. Thereafter, laser system 110 sinters the next layer of powder material 122, and the same layering process continues. Successive layers of sintered powder material 122 eventually result in an additively manufactured preform 140. Importantly, although illustrated as a solid cylindrical structure in FIG. 1, preform 140 may comprise any suitable geometry (e.g., rod, cylinder, plate, bar, or any other symmetrical or non-symmetrical, simple or complex geometry), as based upon the design characteristics of the 3D printing technique.

Additionally, although FIG. 1 illustrates a laser-sintering process to product a preform 140 from powdered feedstocks, alternative additive manufacturing processes may also be utilized. For example, Electron Beam (EBM) additive manufacturing may alternatively be used, where a focused electron beam is used to melt powdered metal in a vacuum chamber (e.g., rather than the laser 115 of FIG. 1.) Similar to laser sintering, the powdered feedstock of Nitinol material is melted and solidified to form a preform.

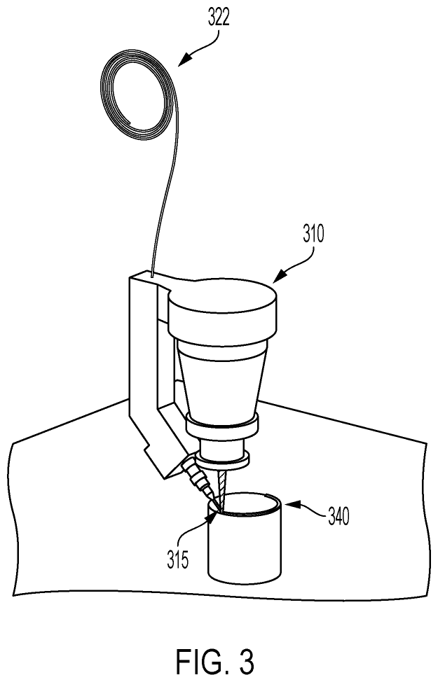

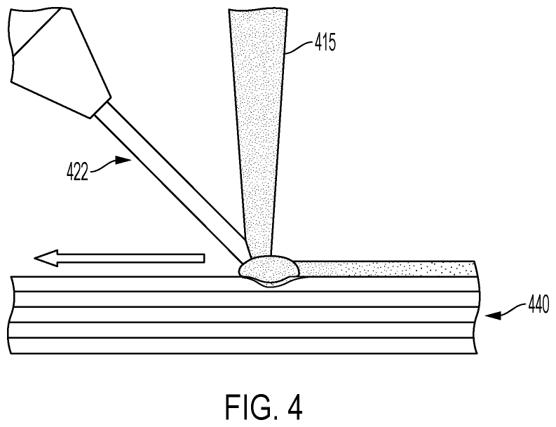

Additionally, and as illustrated in FIGS. 3 and 4, wire-based AM may be used. For example, a wire feedstock 322 may be deposited in a layer-by-layer basis to form the preform 340/440. Here, a heat source 315, such as an electron beam or an arc depositor, melts the wire Nitinol feedstock 322/422, into individual layers of material, resulting in AM preform 340/440. Dissimilar to AM techniques using a powdered feedstock (E.g., laser sintering or EMB), the heat source melts wire filament 322/422 of the Nitinol material and deposits the material layer upon layer to form the preform 340/440.

Therefore, although the present disclosure may specifically indicate certain specific AM techniques, the present disclosure is non-limiting and contemplates other AM techniques that can/could produce similar results to those described herein.

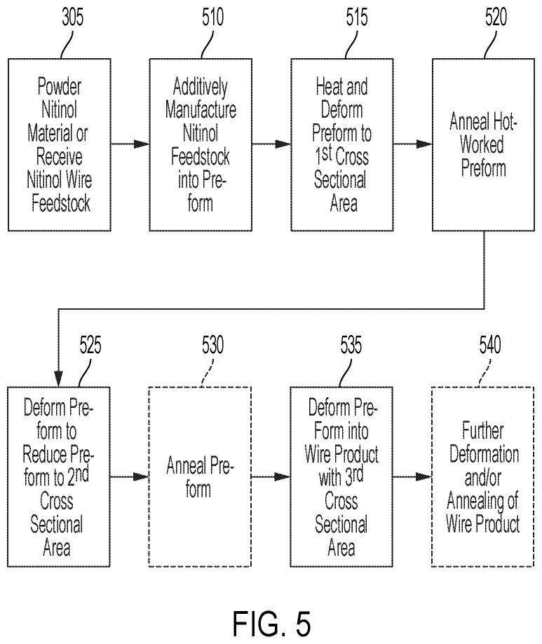

The additive manufacturing of preform 140 (and/or 240, 340, 440) may be used as part of a process for forming a drawn wire of Nitinol material that involves deformation. Such an exemplary method 500 is illustrated in FIG. 5. Here, in a first step 505, as based upon the additive AM utilized (e.g., powder based vs. metal filament/wire based, as described previously) either a metallic wire feedstock is provided, or a metallic powder is created. For example, in a powder-based AM process, the powder may be created utilizing methods that break down bulk metal material, such as Nitinol, into fine particles which may be tailored for applications such as AM including laser sintering. Examples of suitable methods include atomization (e.g., molten metal is broken into droplets using gas or water jets and solidified into powder); milling/crushing (e.g., bulk metal is mechanically ground into powder); chemical reductions (e.g., metal oxides are reduced to powder using hydrogen or carbon); electrolysis (e.g., metal is deposited as powder from an electrolytic solution); plasma methods (e.g., melting metal wire or rods with plasma to produce spherical particles), and the like. Alternatively, metallic filament/wire of Nitinol material may be provided, such as in a solid wire form.

Once the powder is formed and/or wire feedstock is provided, in a second step 510, the metallic material is used as the feedstock in an additive manufacturing process. For example, in a laser sintering additive manufacturing process, a laser melts the powder feedstock material, which is thereafter consolidated to form a preform of the metallic material. Here, the preform may be formed by an AM similar to additive manufacturing system 100, as described with reference to FIGS. 1 and 2. Alternatively, in an electron beam AM process, an electron beam, rather than a laser may be used. Further, in a wire-based additive manufacturing process (E.g., WAAM), such as described with refence to FIGS. 3 and 4, a non-powdered metallic feedstock material may be used to build the preform in a layer-by-layer basis.

Once formed, in a third step 515, the preform is subjected to one or more hot working processes including, but not limited to, forging, extrusion, rolling, swaging, and the like. Here, the preform is heated and deformed, such that the diameter/cross-sectional area/size of the preform material is reduced to an initial (e.g., first) cross-sectional size while at an elevated temperature. In a fourth step 520, annealing occurs after the hot working step, to relieve the internal stresses and restore the ductility of the preform material. In some cases, however, both of steps 515 and 520 may not be used, and the preform object may proceed to cold working steps (e.g., 525) alone. In a fifth step 525, the preform object may be deformed, such as in a cold working process, where the cross-sectional area of the preform is reduced to a second cross-sectional area while at, around, or below room temperature. Exemplary cold working processes include drawing, swaging, rolling, and the like. For example, during a drawing process, the preform may be pulled through a series of progressively smaller dies, and the cross-sectional area of the preform is reduced over the initial and/or hot-worked preform object.

Once cold-worked, the preform may again optionally be annealed, such as in optional step 530, to relieve the internal stresses and restore the ductility of the preform material. Once cold worked (and optionally annealed), in a fifth step 535, the preform may be further deformed to a third cross-sectional area, smaller than the first or second first cross-sectional areas, to form a wire product. Step 535 may be substantially similar to the drawing process of step 525. Thereafter, the wire product may be further annealed and/or further deformed (e.g., similar to steps 530 and 535, respectively) to reduce the diameter of the wire product further. The resulting drawn wire product, formed via additive manufacturing, may have a cross-sectional area or diameter at least 30% less than the original preform.

Traditionally, AM methods produce net or near-net shaped parts by locally melting powder or wire feedstocks to build components layer-by-layer. In some cases, this can be more efficient than conventional “subtractive” machining by minimizing scraping and also allows for geometries not attainable by subtractive methods. However, the larger surface area of powder or wire feedstocks can introduce excess oxygen to the system, potentially increasing inclusion content over conventionally cast Nitinol. Avenues of research related to Nitinol AM include feedstock chemistry and dimensions, melting power and pulse sequences, build directions, and thermal post-processing, with a focus on resultant properties such as chemical and microstructural uniformity, density/porosity, and surface finish. Importantly, AM Nitinol components are seldom, if ever, subjected to wrought deformation processes (e.g., since a key value proposition of AM is to produce in a net or near net shape).

Research in powder- and wire-based AM of shape set and non-shape set memory alloys, such as non-Nitinol alloys, shows two types of retained non-metallic inclusion (NMI) particles: firstly, those present in the parent material, and second, those formed during the AM process. In the second case, the NMIs formed during AM occur at an increased rate over conventional casting techniques because of high surface area starting material with naturally forming reaction layers, and because of trapped species that react during local heating. In non-nitinol alloys, these NMI tend to remain undissolved during melting and are distributed throughout the matrix in sizes exceeding 50 microns. This can cause stress-concentrations leading to increased fatigue damage potency.

However, in Nitinol and Nitinol alloys, NMIs from parent materials (e.g., carbide and/or oxide type), are dissolved or dispersed into the molten parent Nitinol alloy matrix. Both pre-existing, parent NMIs and those formed during AM are dissolved and/or dispersed during local melting. The small melt pool and subsequent rapid solidification of the Nitinol material during AM ensures these NMI remain dissolved and/or finely dispersed throughout the AM preform.

The present invention is based on the discovery that AM Nitinol materials subjected to conventional wrought processing, such as those described with reference to FIG. 5, exhibit extremely fine or even non-detectable inclusions, resulting in excellent performance in terms of structural fatigue, mechanical strength, and ductility. This effect is achieved from the high solidification rate inherent to the AM process and the resultant microstructural fineness. This extension of AM to wrought processing is surprising for several reasons including: (1) presently, AM is intended primarily to form near-net shape components and avoid conventional wrought processing; (2) most variants of AM processing are known to result in the pickup of tramp elements such as oxygen and thus inclusion size refinement or inclusion elimination is not intuitive; and (3) wrought processing of AM-prepared sections adds cost to an already complex manufacturing stream requiring new engineering and process controls to succeed.

Nitinol produced through AM has the benefits of greatly refining and reducing the NMIs that have been found to limit fatigue life. Presently, AM for near net shapes/products occurs without the need for subsequent processing (i.e., heat treatment, internal void reduction with shaped dies or hot-isostatic pressing, or light machining). The present invention aims to advantageously utilize the discovery that greatly refined microstructures are produced from AM, which existing manufacturing processes cannot obtain. Specifically, AM Nitinol allows the microstructural features determined by the solidification time/characteristics to be decoupled from the part size/volume in a way that scaling limitations (i.e. size of preform) is not determined by material performance (i.e. NMI formation) but by machine build volume availability.

Here, it has been surprisingly found that the inclusions in the resulting refined Nitinol wire and/or wire construct may be as large as 30 μm, 25 μm, 15 μm, or 10 μm, or as little as 7 μm, or 5 μm, 3 μm, 2 μm, or 1 μm, or between any of the two foregoing values used as endpoints, such as 30 μm to 1 μm, 10 μm to 1 μm, 5 μm to 1 μm, 3 μm to 1 μm. More specifically, it has been found that the maximum longitudinal inclusion size may be as large as about 20 μm, 18 μm, or 15 μm, or as little 13 μm, 11 μm or 10 μm, or between any of the foregoing value used as endpoints, such as 20 μm to 11 μm, 15 μm to 11 μm or about 11 μm. In this case, the maximum inclusion size may decrease by as much as about 50%, 40%, or 35% or as little 33%, 30% or 25%, as compared with Nitinol wire and/or wire construct manufactured via traditional methods. Inclusions may be measured by several methods known to those of ordinary skill in the art, including optical and electron microscopy, for example according to ASTM F2063.

More specifically, between 90% and 99.5% of the inclusions of the refined wire or wire construct may be less than 10 μm, or less than 5 μm. For instance, about 97%, about 97.5%, about 98%, about 98.5%, about 99%, or 99.5% of the inclusions of the refined wire or wire construct may be less than 5 μm in size. As used herein, the foregoing inclusion size analysis is performed using a lower size cutoff of 1 μm such that inclusions having a size less than 1 μm are excluded from the inclusion count. Because sub-micron features may occur at comparatively high number density, inclusion of features below 1 μm can disproportionately increase the measured fraction of “small” inclusions in count-based statistics. Accordingly, the inclusion percentages recited herein are based on inclusions in a measured size window of from 5 μm to 1 μm (inclusive). Thus, alternatively stated, about 97%, about 97.5%, about 98%, about 98.5%, about 99%, or 99.5% of the inclusions of the refined wire or wire construct may be from 5 μm to 1 μm in size.

Furthermore, it has also been found that the mean area fraction (mean AF; defined as mean AF=mean total area of inclusions/the total cross-sectional area of the material) of such inclusions in the refined Nitinol wire/wire construct may be as high as 3.0%, 2.0%, or 1.5%, or as little as 1.0%, 0.5%, or less than 0.5%, or between any of the forgoing values used as end points, such as from 3.0% to 0.5% or less, from 1.0% to 0.5% of less, or less than 0.5%. For example, the area fraction of the inclusion may be less than 1.0% and/or less than 0.5%.

It has also been found that the maximum area fraction (max AF; defined as max AF=maximum total area of inclusions/the total cross section of the material) of such inclusions in the refined Nitinol wire/wire construct may be as high as 3.0%, 2.0%, or 1.5%, or as little as 1.0%, 0.75%, or less than 0.75%, or between any of the forgoing values used as end points, such as from 3.0% to 0.75% or less, from 1.0% to 0.90% of less, or less than 0.90%. For example, the area fraction of the inclusion may be less than 1.0%.

The relatively small average size and/or maximum size and/or relatively low maximum or mean area fraction of the inclusions results in Nitinol wire having a reduced NMI fatigue damage potency and improves component fatigue durability. Previously, reducing the number of inclusions formed during the AM process has been a significant barrier to the adoption of AM for Nitinol preform production. The present disclosure specifically provides a solution, where AM Nitinol and/or Nitinol alloys are produced substantially “inclusion-free”, and in non-near-net preforms, which can be routed with conventional metal-working (to be described in further detail below) to confer other benefits such as increased strength by cold-working wire, sheet, and tube based semi-finished goods un-achievable with current conventional only methods.

### 6. Processing/Refining of Additively Manufactured Preforms into Wire

The AM preform material (e.g., a rod, plate, hollow tube, etc.) formed by the previously described AM process may be subjected to hot working into the desired preform size and shape. For purposes of the present disclosure, hot working is accomplished by heating the material to an elevated temperature, and particular above the materials crystallization temperature, and performing desired shaping and forming operations while the material is maintained at the elevated temperature. Suitable hot-working processes include, but are not limited to, forging, rolling, and extrusion. During forging, compressive forces is applied to the heated material to shape it into the desired preform dimensions. Forging may create an intermediate billet or rod with uniform dimensions and a refined grain structure, which improves the mechanical properties of the final wire product. Rolling is a continuous deformation process in which the heated material is passed between a series of rotating rolls to progressively reduce its cross-sectional area. In wire production, rolling is used to produce elongated rods with precise dimensions that serve as the preform for further drawing operations. Extrusion involves forcing the heated material through a die to produce a long, uniform preform with a specific cross-sectional shape. This method is particularly useful for creating preforms with complex profiles or consistent properties along the length of the material. These hot-working processes may be used to reduce the cross-sectional area of the preform material, as compared to the cross-sectional area before hot-working.

The hot-worked material may be subjected to an annealing heat treatment prior to cold working. Here, the preform material may be heated and cooled to restore the ductility, relieve residual stresses, refine grain structure, and/or improve machinability of the preform. Specifically, such the hot-worked preform may be heated and held at the target temperature for a specific period to allow for uniform heat distribution and structural changes, such as recrystallization and phase transformations. The preform may then be further cooled, such as in a controlled environment (e.g., furnace or air), to prevent thermal stresses and ensure the desired properties are achieved.

The resulting hot-worked and annealed preform material, such as a billet or rod, may then be further processed into an intermediate form, such as a rod, wire, tube, sheet or plate product by repetitive cold-forming and annealing cycles. For example, a schedule of drawing and annealing of the preform material may to create an initial coarse wire structure ready for final processing. Thereafter, the wires may be subjected to a final cold work conditioning step, and possibly a final heat treatment step, in order to impart desired mechanical properties to the finished wire product as further described below.

Specifically, wire made of a Nitinol (including further alloys thereof, as described herein) may be produced using conventional methods, including a combination of heating, drawing and annealing in order to convert the preform material formed in the AM process into a wire of a desired diameter/cross-sectional area prior to final processing. That is, the preform material is annealed and then drawn through a die or dies to reduce the outer diameter of the intermediate material slightly while also elongating the material, after which the material may be annealed to relieve the internal stresses (i.e., retained cold work) imparted to the material by the drawing process. This annealed material is then drawn through a new die with a smaller finish diameter to further reduce the diameter/cross-sectional area of the material, and to further elongate the material. Further annealing and drawing of the material is iteratively repeated until the material is formed into a wire construct ready for final processing into wire.

For example, a wire of Nitinol may be formed by inserting the preform of Nitinol, as formed by the AM process described previously, into a drawing die. The end protruding through the drawing die is gripped and pulled through the die to reduce the diameter/cross-sectional area of the wire construct. Drawing subjects the wire to cold work. For purposes of the present disclosure, cold-working methods effect material deformation at or near room temperature, e.g., 20-30° C. Drawing imparts cold work, resulting in concomitant reduction in the cross-sectional area. The total cold work imparted to the wire during drawing can be characterized by the following formula (I):

$$c w = 1 - {( \frac{{D}_{2}}{{D}_{1}} )}^{2} \times 100 \% \qquad ( I )$$

wherein “cw” is cold work defined by reduction of the original material area, “D₂” is the outer cross-sectional diameter of the wire after the draw or draws, and “D₁” is the outer cross-sectional diameter of the wire prior to the same draw or draws.

The cold work step may be performed by drawing the wire through a lubricated die having an output diameter D₂, which is less than diameter D₁ of the wire prior to the drawing step. The outer diameter of the wire is accordingly reduced from pre-drawing diameter D₁ to drawn diameter D₂, thereby imparting cold work (cw).

Alternatively, net cold work may be accumulated in the wire by other processes such as cold swaging, rolling the wire (e.g., into a flat ribbon or into other shapes), extrusion, bending, flowforming, severe plastic deformation, or pilgering. Cold work may also be imparted by any combination of techniques including the techniques described here, for example, cold swaging followed by drawing through a lubricated die finished by cold rolling into a ribbon or sheet form or other shaped wire forms. In one exemplary embodiment, the cold work step by which the diameter of the wire is reduced from D₁ to D₂ is performed in a single draw and, in another embodiment, the cold work step by which the diameter of the wire is reduced from D₁ to D₂ is performed in multiple draws which are performed sequentially without any annealing step therebetween. When calculating cold work cw using formula (I) above, it is assumed that no anneal has been performed subsequent to the process of imparting cold work to the material.

For processes where the drawing process is repeated without an intervening anneal of the Nitinol wire, each subsequent drawing step further reduces the cross-sectional area of wire proportionately. Thermal stress relieving, otherwise known in the art as annealing, at a nominal temperature not exceeding the melting point of the wire material is used to improve the ductility of the fully dense composite between drawing steps, thereby allowing further plastic deformation by subsequent drawing steps. Further details regarding wire drawing are discussed in U.S. Pat. No. 7,989,703, issued Aug. 2, 2011, entitled “Alternating Core Composite Wire”, assigned to the assignee of the present invention, the entire disclosure of which is incorporated by reference herein.

Heating the Nitinol wire to a temperature sufficient to cause recovery and recrystallization of grains eliminates accumulated cold work. The cold work imparted by each iterative cold work process is relieved by fully annealing the material between draws, thereby enabling the next iterative cold working process. In full annealing, the cold worked material is heated to a temperature sufficient to substantially relieve the internal stresses stored in the material, thereby relieving the stored cold work and “resetting” cold work to zero.

On the other hand, wires subject to drawing or other mechanical processing without a subsequent annealing process retain an amount of cold work. The amount of retained work depends upon the overall reduction in diameter from D₁ to D₂ and may be quantified by individual grain deformation within the material as a result of the cold work imparted. The wire, in a post-annealing state, may include grains, which may be substantially equiaxed (i.e., grains define generally spheroid shapes in which a measurement of the overall length of the grain is substantially the same regardless of the direction of measurement). After drawing the wire (as described above), the equiaxed grains are converted into elongated grains, such that grains are longitudinal structures defining an elongated grain length G2 and a grain width G3. The elongation of the grains results from the cold working process, with the longitudinal axis of the grains generally aligned with the direction of drawing.

The retained cold work of the Nitinol wire after drawing can be expressed as the ratio of the elongated grain length G2 to the width G3, such that a larger ratio implies a grain which has been “stretched” farther and therefore implies a greater amount of retained cold work. By contrast, annealing the wire after an intermediate drawing process recrystallizes the material, converting elongated grains back to equiaxed grains and “resetting” the retained cold work ratio to 1:1 (i.e., no retained cold work).

For the present Nitinol materials, full annealing (e.g., annealing conducted during the deformation process) may be accomplished at a temperature about 600° C.-900° C. for at least several seconds (e.g., from 2 sec. to 120 sec.) for thin wire (i.e., having a diameter of between 0.0127 mm to 0.8001 mm, corresponding with a cross-sectional area of 0.000127 sq. mm to 0.8001 sq. mm, respectively) to tens of minutes for thicker materials (i.e., having a larger cross-sectional area of between 1 sq. mm and 125 sq. mm). A superelastic anneal (e.g., occurring after the deformation process), can be accomplished at temperatures 450° C.-550° C. and for similar times, again depending on cross-sectional area of the material. Finally, a shape set anneal, imparting permanent shape to the wire product/constant, may occur at similar times and temperatures. Of course, in each of the foregoing cases, a relatively higher temperature annealing process can utilize a relatively shorter time to achieve a full anneal, while a relatively lower temperature will typically utilize a relatively longer time to achieve a full anneal. Additionally, annealing parameters are expected to differ based on wire diameters. Smaller diameters typically reduce the annealing time required at a given temperature. Whether a full anneal has been accomplished can be verified in a number of ways well known in the art, such as microstructural examinations using scanning electron microscopy (SEM), mechanical testing for ductility, strength, elasticity, etc., and other methods.

As a result of the deformation and annealing processes, the cross-sectional area of the preform may be reduced, as compared with the initial cross-sectional area of the preform formed by the additive manufacturing process described previously, by as little as 25%, 30%, 40% 50%, or 60%, or as much as 75%, 80%, 85%, or even 90%, 95%, or 99%, or between any of the two foregoing values used as endpoints, such as 25% to 99%, 30% to 99%, and/or 75% to 99%.

Although described previously as a solid wire formed entirely of Nitinol materials, the disclosure is non-limiting. For example, the Nitinol material may be incorporated as part of a wire construct, such as a DFT® wire construct including a shell and a core. In this case, either the shell or the core, or both, may be formed of Nitinol and/or Nitinol based alloy(s). In this case, the drawing and annealing process, described previously, many be performed in substantially the same way, resulting in the same, or substantially similar overall wire diameter/cross-sectional area reductions as described previously.

The resulting coarse wire material may then be finally processed into a final form, such as a fine wire suitable for integration into a stent or other medical device. Exemplary wire constructs are described in further detail below.

### 7 Nickel Titanium (NiTi) Alloy Materials

Metallic materials comprising nickel titanium (NiTi), such as Nitinol and/or Nitinol alloys of the present disclosure, are described herein. Here, the nickel titanium materials may be regarded as shape-set alloy materials, that can return to a predefined shape when heated above a certain temperature.

In any of the embodiments of the present disclosure, the materials listed in either of Tables 1 and 2 below may be used as the feedstock material in the AM process, and the resulting preform and wire products/constructs may comprise any of the materials listed in Table 1 and 2 below. Particularly, the materials of Table 1 and Table 2 below may be used as the feedstock powder in a laser sintering AM or may be used as the metallic feedstock in a WAAM process. The resulting wire or wire construct may therefore comprise any composition defined in tables 1 and 2 below.

Standard specifications and chemistry for nickel titanium (e.g., “NiTi”) binary alloy for use in surgical implant applications may be found in ASTM F2063, the entire disclosure of which is hereby incorporated herein by reference. A material constituency for materials made in accordance with ASTM F2063 is shown below as Table 1.

TABLE 1
Chemical Composition Requirements of NiTi

| Element | % (mass/mass) |
|---|---|
| Nickel | 54.5 to 57.0 |
| Carbon, maximum | 0.040 |
| Cobalt, maximum | 0.050 |
| Copper, maximum | 0.010 |
| Chromium, maximum | 0.010 |
| Hydrogen, maximum | 0.005 |
| Iron, maximum | 0.050 |
| Niobium, maximum | 0.025 |
| Nitrogen, maximum | 0.005 |
| Oxygen, maximum | 0.040 |
| Titanium^{A} | Balance |

^{A}Approximately equal to the difference between 100% and the sum percentage of the other specified elements. The percentage titanium content by difference is not required to be reported.

Table 2 lists additional shape memory alloys of NiTi alloy. Specifically, ternary alloys comprise the NiTi composition of Table 1, with the additional third (e.g., ternary) and optionally, fourth (e.g., quaternary) components, as indicated in Table 2 below. For example, “NiTi #2” designates a composition including NiTi, with further inclusion of the designated ternary element Cr at 0.25%.

TABLE 2
Listing of NiTi ternary alloys

| Name | Content (Atomic %) |
|---|---|
| NiTi#2 | 0.25% Cr; balance NiTi |
| NiTi#3 | 1.2% Co; balance NiTi |
| NiTi-Y | 0.075% Y; balance NiTi |
| NiTi-Nb | 4-11% Nb; balance NiTi |
| NiTi-NbY | 4-11% Nb and 0.03-0.1% Y; balance NiTi |
| NiTi-Cu | 6-7% Cu, balance NiTi |
| NiTi-Fe | 3.5% Fe, balance NiTi |
| NiTi-Hf | 5-20% Hf; balance NiTi |
| NiTi-Zr | 3-20% Zr; balance NiTi |
| NiTi-Pt | 5-30% Pt; balance NiTi |
| NiTi-Ta | 1-10% Ta; balance NiTi |

### 8. Physical Properties of Refined Wire/Wire Construct

As described previously, the present disclosure regards a wire or wire construct formed of Nitinol or Nitinol alloy and manufactured in additive manufacturing process. The AM of Nitinol results in little to no inclusions in the refined wire product (e.g., 90% of the inclusions of the refined wire or wire construct may be from 25 μm to 1 μm, from 10 μm to 1 μm, and/or from 5 μm to 1 μm). This in turn results in higher physical performance characteristics than conventionally manufactured wire products of Nitinol, such as excellent performance in terms of structural fatigue, mechanical strength, and ductility.

In constant force thermal cycle fatigue (i.e. actuation fatigue) in accordance with the Examples herein, a wire sample typically 100 mm long is hung with a weight crimped to the bottom end of the wire and Joule heated while under constant load. A position sensor monitors the location of the weight and stops the current when the appropriate deformation for the thermal cycle is reached. The thermal cycle is repeated until fracture, or a predetermined number of cycles is achieved for runout, 50,000 to 150,000 thermal cycles.

In rotary beam fatigue testing in accordance with the Examples herein, a wire sample is cut to a length (e.g. about 118 mm for a 0.33 mm diameter wire or about 68 mm for a 0.127 mm diameter wire), then secured at its axial ends to rotatable jaws. The free portion of the wire between the jaws is bent to introduce a desired tensile strain at the “peak” or outermost portion of the bend. Directly opposite this peak of the bend, the wire experiences a compressive strain equal to the tensile strain, with the nominal value of both the tensile and compressive strains referred to herein as the “strain amplitude.” The jaws are then rotated in concert (i.e., each jaw rotated with the same speed and in the same direction), such that the area of maximum tensile strain is rotated around the wire “peak” and transitioned to the area of maximum compressive strain with each 180-degree rotation of the jaws and wire. Rotary beam fatigue testing is further described in ASTM E2948-14, the entire disclosure of which is hereby expressly incorporated herein by reference.

The fatigue strength and endurance of the AM refined Nitinol wire product, as performed on the rotary beam described above, when executed at a 1% strain amplitude and room temperature, may be based upon the type of Nitinol alloy material utilized. For Example, a binary Nitinol alloy, such as those described with reference to Table 1 above, may have a higher fatigue strength and endurance than a ternary or greater alloy of Nitinol.

In the case of a Binary alloy of Nitinol, such as those described in table 1 above, the fatigue strength and endurance can be as little as about 1,000,000 cycles, 2,000,000 cycles, or 5,000,000 cycles, or as great as about 10,000,000 cycles, 50,000,000 cycles, or 100,000,000 cycles before failure with a 1% strain amplitude at room temperature. For example, the fatigue strength/endurance of the AM wire comprising a binary alloy of Nitinol may be greater than 1,000,000 cycles, greater than 10,000,000 cycles, and/or about 100,000,000 cycles when performed with a 1% strain amplitude at room temperature.

In the case of a ternary or greater alloy of Nitinol, such as those describe with reference to Table 2 above, the fatigue strength and endurance can be as little as about 5,000 cycles, 10,000 cycles, or 15,000 cycles, or as great as about 20,000 cycles, 50,000 cycles, or 100,000 cycles before failure with a 1% strain amplitude at room temperature. For example, the fatigue strength/endurance of the AM Nitinol wire comprising the ternary or greater alloy may be greater than 10,000 cycles, greater than 20,000 cycles, about 50,000 cycles or about 100,000 cycles when performed with a 1% strain amplitude at room temperature.

Additionally, or alternatively, when performing “run out” until 107 (e.g., 10,000,000) cycles at room temperature, the Nitinol wire may be able to endure 107 cycles at a strain amplitude as low as 0.5%, 0.8% or 0.9%, or as high as 1.0%, 1.1% or 1.2% before failure. In other words, the AM Nitinol wire may be able to endure 107 cycles before failure at an amplitude range from 0.8% strain to 1.2% strain.

This enhanced fatigue endurance of the present AM Nitinol wire enables the alloy to be used in in vivo applications such as stents, including vascular (e.g., cardiac) and non-vascular (e.g., gastrointestinal, urinary) stents. Because alloys used in stents and similar in vivo applications may undergo many cycles, the present wire is ideally suited for use in stents implanted in high-flexion areas (i.e., extremities) and other demanding applications. Specifically, the AM Nitinol wire may be used in neurovascular stents such as exemplified in Example 9; peripheral wire form stents such as exemplified in Example 10; coil assist stents such as exemplified in Example 11, septal occluders such as exemplified in Example 12; cables used in biostimulation devices such as exemplified in Example 13; heart valve frames such as exemplified in Example 14; laser cut tube stents for aortic grafting such as exemplified in Example 15; and laser cut sheets for hernia repair grafts such as exemplified in Example 16.

### EXAMPLES

The following non-limiting Examples illustrate various features and characteristics of the present invention, which is not to be construed as limited thereto.

### Example 1A

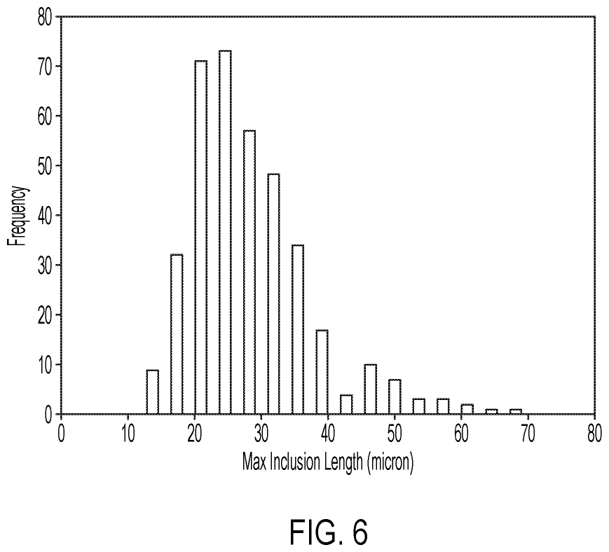

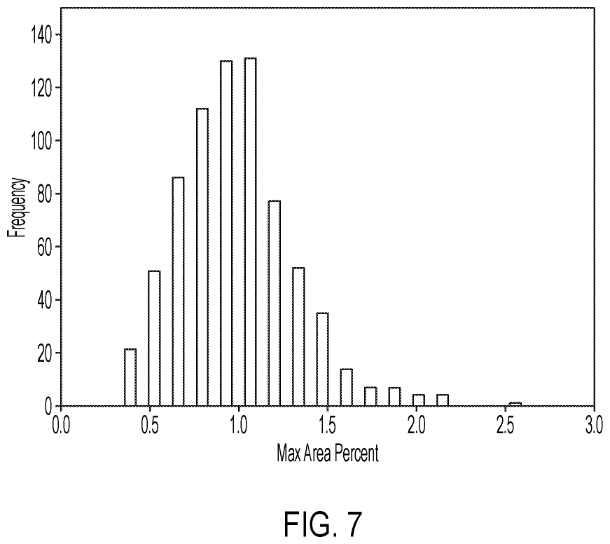

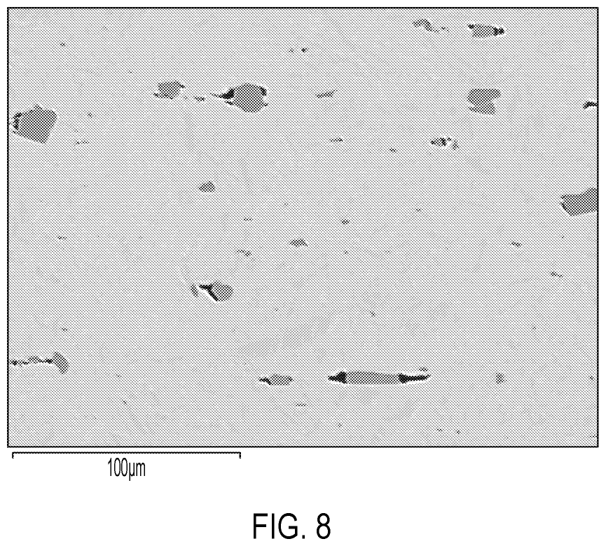

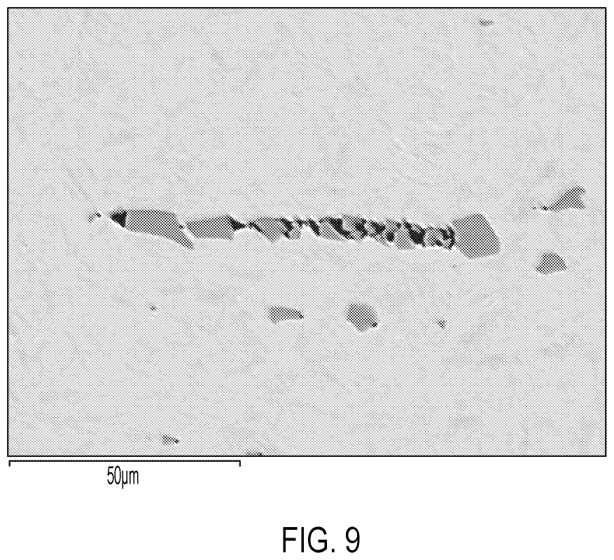

Nitinol bars 63.5 mm in diameter were manufactured with conventional melt, hot working, and machining practices and atomized into powder with an EIGA free-fall atomization process and sieved into 20-63 μm and 50-150 μm nominal sizes suitable for AM. FIG. 6 is a histogram of the maximum length of nonmetallic inclusion void assemblies for traditionally manufactured NiTi #5. FIG. 6 shows that the maximum length of the nonmetallic inclusion-void assemblies for the traditionally manufactured NiTi #5 is commonly between 18.5 μm to 48.4 μm for the middle 89% of maximums measured. FIG. 7 is a histogram of the maximum area percent of nonmetallic inclusion void assemblies for traditionally manufactured NiTi #5. FIG. 7 shows that the maximum area percent is commonly between 0.57% to 1.56% for the middle 89% of areas measured. FIGS. 8 and 9 show micrographs with the characteristic level of cleanliness produced from traditional manufacturing methods for NiTi #5, as demonstrated in FIGS. 6 and 7.

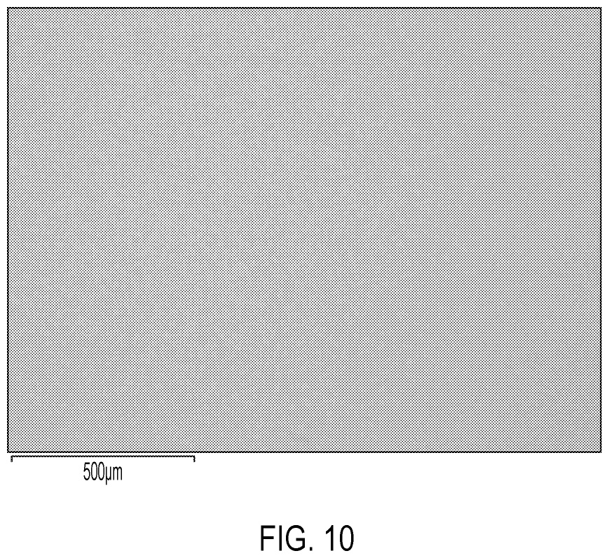

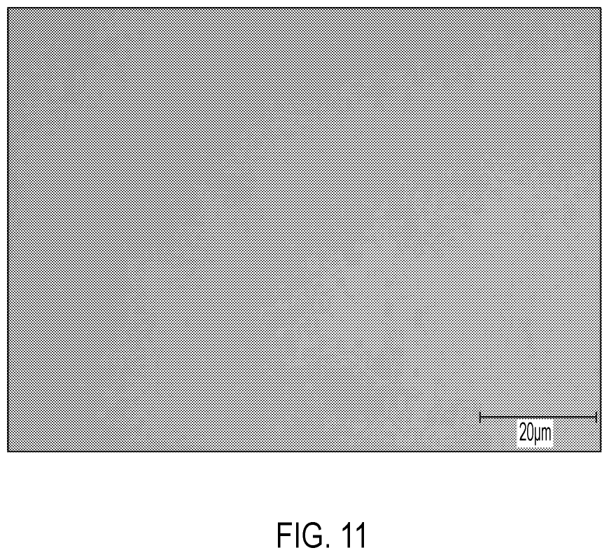

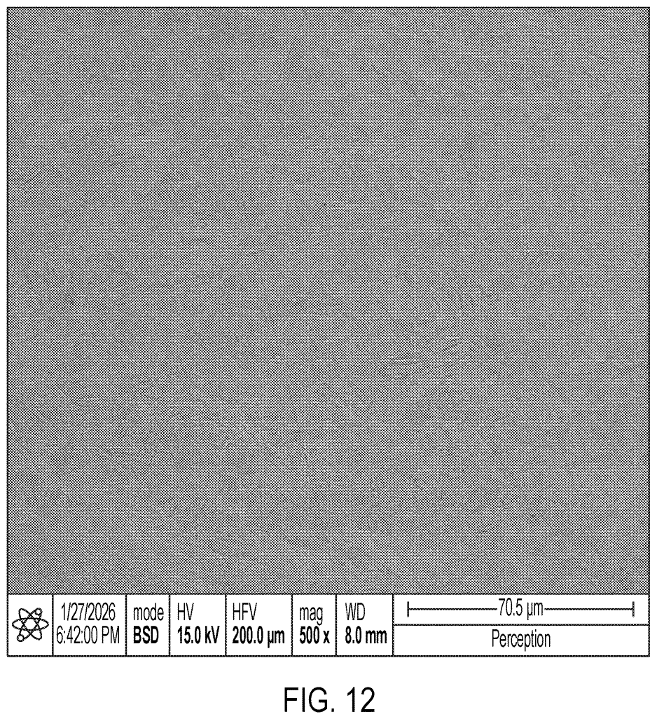

Next, preforms were additively manufactured from the 20-63 μm nominal particle size Nitinol #5 powder using a Renishaw AM250 pulsed laser powder bed fusion (LPBF) and EOS M290 LPBF systems with volumetric energy densities of 53 J/mm³ and 44.4 J/mm³ respectively. The 9.53 mm diameter by 174.63 mm long Renishaw preforms were hot swaged at 850° C. to 6.35 mm and 3.30 mm diameters and cold drawn to 2.16 mm and 0.025 mm diameter wire sizes for testing. The EOS preforms were printed to 12.7 mm square by 152.4 mm long and hot rolled at 850° C. to 4.52 mm diameter. FIG. 9 is a histogram of the maximum area percent of nonmetallic inclusion void assemblies for traditionally manufactured NiTi #5. FIGS. 10 and 11 show inclusion free micrographs of the 3.30 mm diameter Renishaw printed wire at 80× magnification and 2.16 mm diameter Renishaw printed wire at 1,000× magnification respectively showing 0.0 μm nonmetallic inclusion void assembly maximum length and 0.00% maximum area. FIG. 12 also shows an inclusion free micrograph of the 4.52 mm diameter EOS printed wire at 500×0.0 μm nonmetallic inclusion void assembly maximum length and 0.00% maximum area, demonstrating microstructure cleanliness that can be achieved by manufacturing wrought Nitinol beginning with AM.

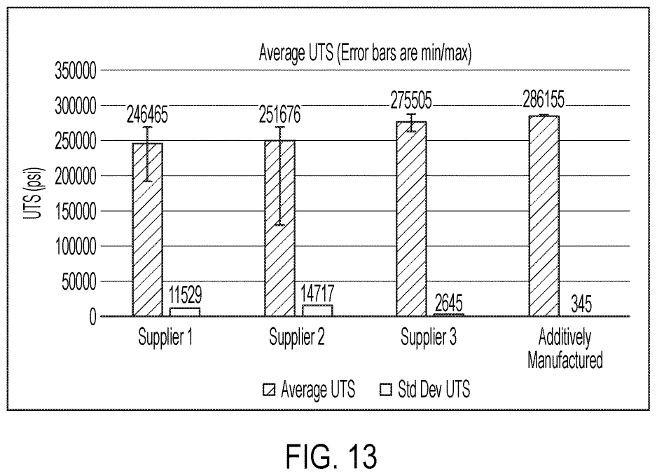

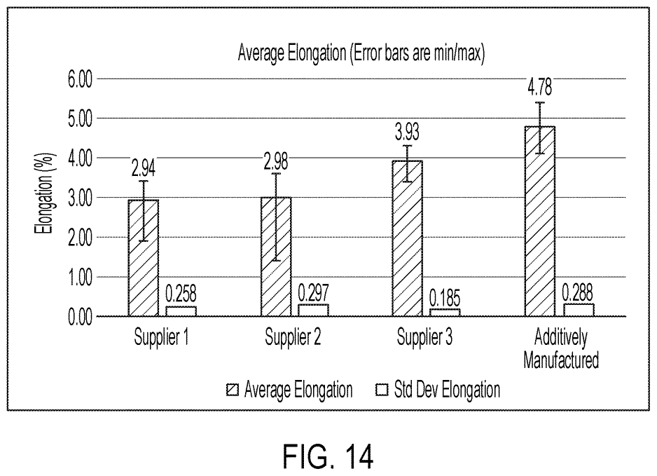

FIGS. 13 and 14 compare the average ultimate tensile strength (UTS) and elongation of 30 breaks from different Nitinol suppliers of 0.025 mm diameter wire. The Nitinol wire manufactured from Renishaw AM preforms (e.g., indicated as “Present Invention” in FIGS. 11 and 12) had the highest average tensile strength and lowest standard deviation. The tensile strength exhibited by the wire manufactured from AM preforms was 10 to 35 KSI higher than the Nitinol wire made from currently available market suppliers. Additionally, the AM preform also exhibited the highest average elongation, 25 to 60% higher than what was achieved by Nitinol wires made from current available market suppliers.

### Example 1B

The wire of Example 1A is cold drawn to 0.0127″ diameter with conventional methods. The wire is then rotary beam fatigue tested at 1% strain and all samples exhibit run outs to 10 million cycles.

### Example 2

A conventionally cast and hot forged binary NiTi bar is gas atomized into powder and sieved for additive manufacturing. A preform is printed from the nominal 15-60 μm powder via laser powder bed fusion to 4″ diameter, hot forged to 2″ diameter, and hot rolled to 0.25″ at 800-900° C. At 0.25″, the microstructure had <5 micron max inclusion and <0.5% area fraction of inclusions. It is then conventionally cold drawn via wire drawing to 0.0127″ diameter. Then it is heat treated and rotary bend fatigue tested at 1.2% zero-mean alternating strain. The wire exhibits run out to 10 million cycles.

### Example 3

A conventionally cast and hot forged binary NiTi bar was gas atomized into powder and sieved for additive manufacturing. A preform was printed from the nominal 50-150 μm powder via electron beam powder bed fusion to 4″ diameter, hot forged to 2″ diameter, and hot rolled to 0.25″ at 800-900° C. At 0.25″, the microstructure had <5 micron max inclusion and <0.5% area fraction of inclusions. It was conventionally cold drawn via wire drawing to 0.0127″ diameter. Then it was heat treated and rotary bend fatigue tested at 1.2% zero-mean alternating strain. The wire ran out to 10 million cycles.

### Example 4

Conventionally cast and hot forged binary NiTi was conventionally wire drawn to 0.040″ diameter. From this wire, Wire Arc Additive Manufacturing was employed using a Meltio M600 set to 750 W to produce a 0.63″ diameter preform. The preform was hot extruded at 850° C. to 0.2″ diameter and then conventionally cold drawn to 0.005″ diameter.

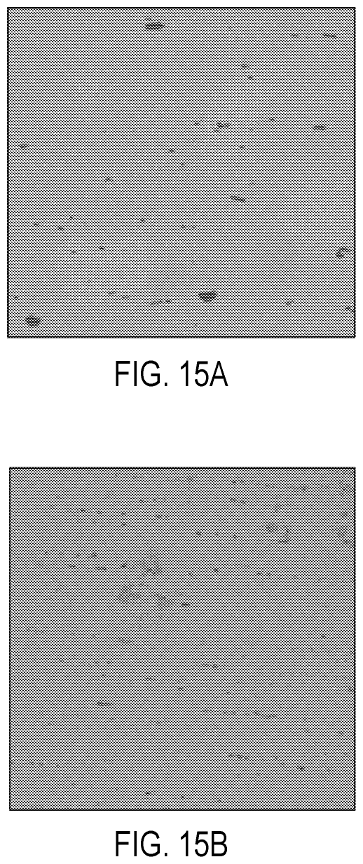

The microstructure of the inventive wire was evaluated for inclusions at 0.2″ diameter and compared to the conventional wire used as input. The results of this evaluation are shown in Table 3 below as well as and FIGS. 15A and 15B. Specifically, FIG. 15A illustrates a Longitudinal view of conventionally produced nitinol wire input and FIG. 15B illustrious a Longitudinal view of inventive additively manufactured and drawn wire. Importantly, the inclusion size analysis is conducted using a lower size cutoff of 1 μm, and therefore, the percentage of 5 μm of greater excludes inclusions having a size less than 1 μm. Such sub-micron features can be present at comparatively high counts and may disproportionately influence count-based inclusion statistics (e.g., exaggerating the count of inclusions sub 5 μm, thus conflating the percentage of 5 μm to a lower/lesser amount). Accordingly, the inclusion percentages recited herein are based on inclusions in a measured size window of 1 μm to 5 μm (inclusive). While the total area percent of the inclusions was unchanged, the maximum size and total size distribution were substantially reduced in the current invention.

TABLE 3
AM Wire vs. Conventional Wire Performance

| | Conventional Wire | Inventive |
|---|---|---|
| Parameter | Input | Example |
| Max longitudinal inclusion size (μm) | 20.8 | 13.6 |
| Inclusions > 5 μm ¹ | 15.20% | 1.05% |
| Max Inclusion Area (%) | 0.89 | 0.94 |
| Average Inclusion Area (%) | 0.46 ± 0.17 | 0.51 ± 0.12 |

¹ Inclusions of less than 1 μm or less are not taken into account in calculating the percentage of inclusions of 5 μm or greater.

To assess fatigue performance improvement, the inventive 0.005″ wire and conventionally processed 0.005″ wire were both heat treated at 500° C. for 5 minutes and then rotary-beam-fatigue tested at 37° C. and 3600 rpm at 1% zero mean strain. The results of this assessment showing the cycles to failure of each specimen are compared in Table 4. The inventive wire achieved 10 million cycle runouts in 9 of 10 specimens while the conventionally processed wire failed to achieve 10 million cycle runouts in any of the 10 specimens.

TABLE 4
Cycles to failure of each 0.005″ diameter wire
rotary beam fatigue tested at 1% zero mean strain.

| | | Present |
|---|---|---|
| | Conventional | Inventive |
| Specimen | Wire | Wire |
| 1 | 14,947 | 10,000,000 |
| 2 | 12,839 | 10,000,000 |
| 3 | 18,262 | 10,000,000 |
| 4 | 9,448 | 16,201 |
| 5 | 9,474 | 10,000,000 |
| 6 | 10,587 | 10,000,000 |
| 7 | 11,435 | 10,000,000 |
| 8 | 13,173 | 10,000,000 |
| 9 | 14,312 | 10,000,000 |
| 10 | 11,471 | 10,000,000 |

### Example 5

Conventionally cast and hot forged binary NiTi is gas atomized, sieved to 15-60 μm nominal particle size, and printed via LPBF to a 0.5″×4″×12″ preform sheet. The preform is hot and cold rolled to 0.010″ thickness, having a microstructure with <5 micron max inclusions and <0.5% area fraction of inclusions. The formed sheet is heat treated to introduce superelasticity. Fatigue specimens are cut via laser from a sheet and fatigue tested at 1.2% alternating strain. The fatigue specimens run out to 10 million cycles.

### Example 6

Conventionally cast and hot forged binary NiTi is gas atomized, sieved to 50-150 μm nominal particle size, and printed via electron beam powder bed fusion to a preform tube of 1″ OD and 0.75″ ID×24″ length. The preform is hot worked and cold drawn to form tubing with 0.125″ OD and 0.1″ ID and having a microstructure with <5 micron max inclusion and <0.5% area fraction of inclusions. The tubing is heat treated to introduce superelasticity. Fatigue specimens are cut via laser from the tube and fatigue tested at 1.2% alternating zero mean strain, exhibiting a run-out to 10 million cycles.

### Example 7

Conventionally cast and hot forged NiTiNbY is gas atomized, sieved to 50-150 μm nominal particle size, and printed via electron beam powder bed fusion to a 4″ diameter preform, which is then hot forged to 2″ diameter, and then hot rolled to 0.25″ diameter at 800-900° C. At 0.25″ diameter, the preform has a microstructure with <5 micron max inclusion and <0.5% area fraction of inclusions. Wire is conventionally cold drawn from the preform to 0.0127″ diameter, then is heat treated and rotary bend fatigue tested at 0.9% alternating zero mean strain, exhibiting a run-out to 10 million cycles.

### Example 8

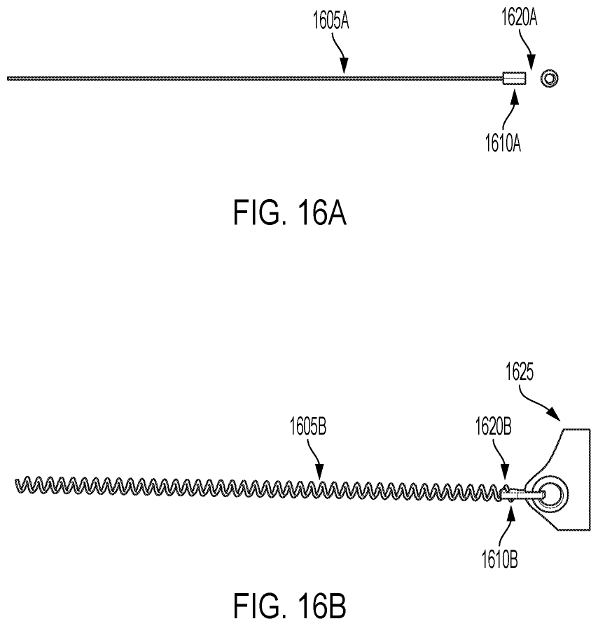

Nitinol wire is made by additively manufacturing a billet and then conventionally working it down into fine wire with a diameter of 0.003″. As illustrated in FIGS. 16A and 16B, the wire 1605A/B is thermomechanically trained such that it exhibits stable thermomechanical actuation suitable for use as an actuator. Fittings 1620 are crimped onto the ends (1610 and unillustrated) of the wire 1605 for transmitting both electrical current to resistively heat the wire 1605 and mechanical load. The actuator wire 1625 is able to move an applied load of 350 MPa with a strain of 3.5% for 50,000 thermal cycles.

### Example 9

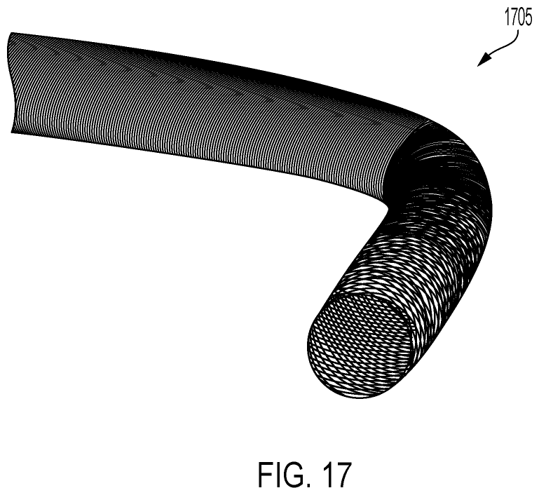

Nitinol wire is made by additively manufacturing a tube. The tube is filled with radiopaque platinum then drawn together to form a DFT composite. The composite is then conventionally worked into fine wire with a diameter of 0.001″. This wire is cut into 48 pieces which are braided together onto a 2, 3, 4, or 5 mm mandrel. The braid 1705, as illustrated in FIG. 17 is heat treated at 500° C. for 10 minutes and then removed from the mandrel. The braid is cut into lengths of 10 to 50 mm to serve as flow diversion devices, such as neurovascular stents. The reduced inclusion count in the inventive wire improves device lifetime and prevents fractures.

### Example 10

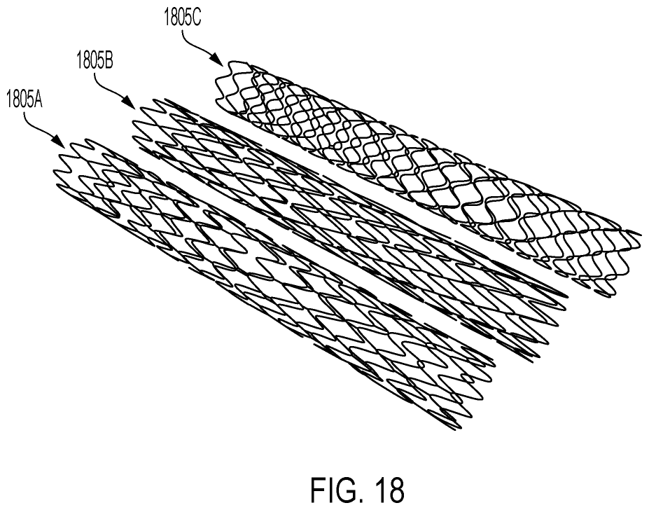

Nitinol wire is made by additively manufacturing a billet and then conventionally working it down into fine wire with a diameter of 0.005″. The wire is wrapped onto a mandrel in a zig-zag pattern. The wire and mandrel are heat treated at 500° C. for 10 minutes and then the wire is removed from the mandrel to form a wire-form stents 1805A, 1805B, 1805C suitable for use in peripheral vascular devices, as illustrated in FIG. 18. The reduced inclusion count in the inventive wire improves device lifetime and prevents fractures.

### Example 11

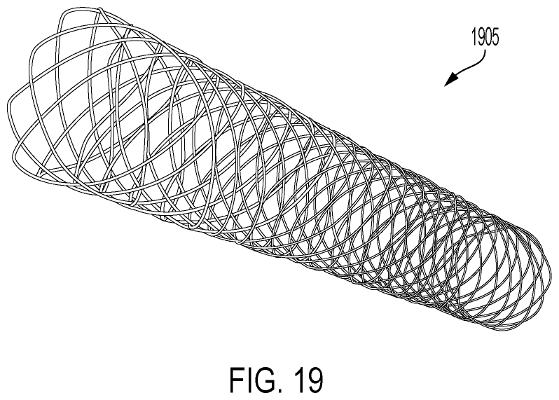

Nitinol wire is made by additively manufacturing a billet and then conventionally working it down into fine wire with a diameter of 0.003″. The wire is braided onto a mandrel in a single-wire closed end design. The wire and mandrel is heat treat at 500° C. for 10 minutes and then the wire is removed from the mandrel to produce a coil assist stent 1905, as illustrated in FIG. 19. This stent assists closure of wide-necked aneurysms by preventing occlusion coils from falling into the artery lumen. The reduced inclusion count in the inventive wire improves device lifetime and prevents fractures.

### Example 12

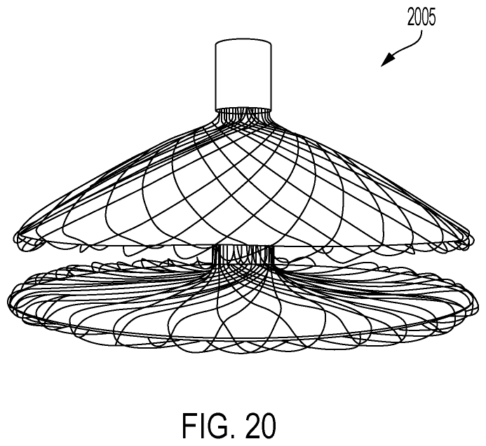

Nitinol wire is made by additively manufacturing a billet and then conventionally working it down into fine wire with a diameter of 0.004″. The wire is braided onto a mandrel, compressed axially, and then heat treated at 500° C. for 10 minutes to produce a septal occlusion device 2005, as illustrated in FIG. 20. The reduced inclusion count in the inventive wire improves device lifetime and prevents fractures.

### Example 13

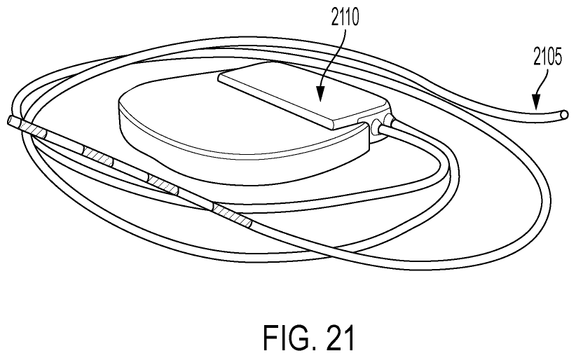

Nitinol wire is made by additively manufacturing a tube. The tube is filled with conductive silver then drawn together to form a DFT composite. The composite is then conventionally worked into fine wire with a diameter of 0.001″. This wire is formed into a 7×7 cable, heat treated, and jacketed with an insulative ETFE polymer. The cable 2105 is then incorporated into a biostimulation device 2101 to transmit electrical signals, as illustrated in FIG. 21. The reduced inclusion count in the inventive wire improves device lifetime and prevents fractures.

### Example 14

Nitinol tubing is made by additively manufacturing a tube. The tube is conventionally drawn by tube drawing to a size of 9 mm OD and 8 mm ID. This tube is laser cut, iteratively expanded and heat treated, and finally electropolished to form a heart valve frame. The reduced inclusion content in the inventive tubing improves device lifetime and prevents fractures.

### Example 15

Nitinol tubing is made by additively manufacturing a billet. The billet is conventionally forged to 30 mm diameter bar. The bar is then conventionally gun drilled and drawn into tubing with an outer diameter of 3 mm and a wall thickness of 0.15 mm. The tube is laser cut, iteratively expanded and heat treated, and finally electropolished to form a an aortic graft frame. The reduced inclusion content in the inventive tubing improves device lifetime and prevents fractures.

### Example 16

Nitinol sheet is made by additively manufacturing a slab. The slab is conventionally hot and cold rolled to a sheet with a final thickness of 0.004″. The sheet is heat treated at 500° C. for 15 minutes to impart superelasticity. The sheet is then laser cut to produce a thin mesh for hernia repair. The reduced inclusion content in the inventive sheet improves device lifetime and prevents fractures.

### ASPECTS

Aspect 1 is a method of producing a wire product from a nickel-titanium shape memory alloy, comprising consolidating a nickel-titanium shape memory alloy to a preform using an additive manufacturing process, the preform having a first cross-sectional area, and deforming the preform to manufacture a wire product having a second cross-sectional area, the second cross-sectional area being at least 30% less than the first cross-sectional area.

Aspect 2 is the method of Aspect 1, further comprising heating and deforming the preform to reduce the cross-sectional area of the preform.

Aspect 3 is the method of any one of Aspects 1 through 2, further comprising annealing the preform or wire.

Aspect 4 is the method of any one of Aspects 1 through 3, wherein the deforming comprises at least two deformation processes selected from forging, extrusion, drawing, swaging, and rolling.

Aspect 5 is the method of any one of Aspects 1 through 4, wherein the consolidating comprises melting a nickel-titanium shape memory alloy provided in powder form and allowing the nickel-titanium shape memory alloy to solidify to the preform.

Aspect 6 is the method of any one of Aspects 1 through 4, wherein the consolidating comprises melting a nickel-titanium shape memory alloy provided in wire form and allowing the nickel-titanium shape memory alloy to solidify to the preform.

Aspect 7 is the method of any one of Aspects 1 through 6, wherein the deforming achieves a cross-sectional area reduction such that the second cross-sectional area is at least 75% less than the first cross-sectional area.

Aspect 8 is the method of any one of Aspects 1 through 7, wherein the deforming achieves a cross-sectional area reduction such that the second cross-sectional area is at least 99% less than the first cross-sectional area.

Aspect 9 is the method of any one of Aspects 1 through 8, wherein the nickel-titanium shape memory alloy is a binary or ternary alloy.

Aspect 10 is the method of any one of Aspects 1 through 9, wherein the wire product comprises at least 96% of all total visible inclusions having a longitudinal maximum dimension from 5 microns to 1 micron, and a rotary beam fatigue strength exceeding 10,000 cycles at 1% strain.

Aspect 11 is the method of any one of Aspects 1 through 10, wherein the wire product comprises at least 98.5% of all total visible inclusions having a longitudinal maximum dimension of from 5 microns to 1 micron.

Aspect 12 is the method of any one of Aspects 1 through 11, wherein the wire product comprises a rotary beam fatigue strength exceeding 5,000,000 cycles at 1% strain.

Aspect 13 is the method of any one of Aspects 1 through 12, wherein the wire product comprises a rotary beam fatigue strength exceeding 10,000,000 cycles at 1% strain.

Aspect 14 is a wire product formed of a nickel-titanium shape memory alloy comprising at least 96% of all total visible inclusions having a longitudinal maximum dimension of from 5 microns to 1 micron, and a rotary beam fatigue strength exceeding 10,000 cycles at 1% strain.

Aspect 15 is the wire product of Aspect 14, comprising at least 98.5% of all total visible inclusions having a longitudinal maximum dimension of from 5 microns to 1 micron.

Aspect 16 is the wire product of any one of Aspects 14 through 15, comprising a rotary beam fatigue strength exceeding 5,000,000 cycles at 1% strain.

Aspect 17 is the wire product of any one of Aspects 14 through 16, comprising a rotary beam fatigue strength exceeding 10,000,000 cycles at 1% strain.

Aspect 18 is a method of producing a wire product from a nickel-titanium shape memory alloy, comprising consolidating a nickel-titanium shape memory alloy to a preform using an additive manufacturing process, the preform having a first cross-sectional area, heating the preform, deforming the heated preform to reduce the first cross-sectional area of the preform to a second cross-sectional area, annealing the deformed preform, and deforming the preform at least once to form a wire product having a third cross-sectional area, wherein the third cross-sectional area is at least 30% less than the first cross-sectional area.

Aspect 19 is a medical device formed from the wire product of Aspect 18.

Aspect 20 is the medical device of Aspect 19, wherein the medical device is one of a vascular stent, a non-vascular stent, a neurovascular stent, a peripheral wire-form stent, a coil-assist stent, a septal occluder, a cable for a biostimulation device, a heart valve frame, a laser-cut tube stent for aortic grafting, or a laser-cut sheet for hernia repair graft.

What is claimed is:

1. A method of producing a wire product from a nickel-titanium shape memory alloy, comprising:
consolidating a nickel-titanium shape memory alloy to a preform using an additive manufacturing process, the preform having a first cross-sectional area; and
deforming the preform to manufacture a wire product having a second cross-sectional area, the second cross-sectional area being at least 30% less than the first cross-sectional area.

2. The method of claim 1, further comprising heating and deforming the preform to reduce the cross-sectional area of the preform.

3. The method of claim 1, further comprising annealing the preform or wire.

4. The method of claim 1, wherein the deformation step comprises at least two deformation processes selected from forging, extrusion, drawing, swaging, and/or rolling.

5. The method of claim 1, wherein the consolidation step comprises melting a nickel-titanium shape memory alloy provided in powder form and allowing the nickel-titanium shape memory alloy to solidify to the preform.

6. The method of claim 1, wherein the consolidation step comprises melting a nickel-titanium shape memory alloy provided in wire form and allowing the nickel-titanium shape memory alloy to solidify to the preform.

7. The method of claim 1, wherein the deformation step achieves a cross-sectional diameter reduction wherein the second cross-sectional area is at least 75% less than the first cross-sectional area.

8. The method of claim 1, wherein the drawing step achieve a cross-sectional diameter reduction wherein the second cross-sectional area is at least 99% less than the first cross-sectional area.

9. The method of claim 1, wherein the nickel-titanium shape memory alloy is a binary or ternary alloy.

10. The method of claim 1, wherein the wire product comprises:
at least 96% of all total visible inclusions having a longitudinal maximum dimension from 5 microns to 1 micron; and
a rotary beam fatigue strength exceeding 10,000 cycles at 1% strain.

11. The method of claim 10, wherein the wire product comprises at least 98.5% of all total visible inclusions having a longitudinal maximum dimension of from 5 microns to 1 micron.

12. The method of claim 10, wherein the wire product comprises a rotary beam fatigue strength exceeding 5,000,000 cycles at 1% strain.

13. The method of claim 12, wherein the wire product comprises a rotary beam fatigue strength exceeding 10,000,000 cycles at 1% strain.

14. A wire product formed of a nickel-titanium shape memory alloy comprising:
at least 96% of all total visible inclusions having a longitudinal maximum dimension of from 5 microns to 1 micron; and
a rotary beam fatigue strength exceeding 10,000 cycles at 1% strain.

15. The wire product of claim 14, comprising at least 98.5% of all total visible inclusions having a longitudinal maximum dimension of from 5 microns to 1 micron.

16. The wire product of claim 14, comprising a rotary beam fatigue strength exceeding 5,000,000 cycles at 1% strain.

17. The wire product of claim 14, comprising a rotary beam fatigue strength exceeding 10,000,000 cycles at 1% strain.

18. A method of producing a wire product from a nickel-titanium shape memory alloy, comprising:
consolidating a nickel-titanium shape memory alloy to a preform using an additive manufacturing process, the preform having a first cross-sectional area;
heating the preform;
deforming the heated preform to reduce the first cross-sectional area of the preform to a second cross-sectional area;
annealing the deformed preform; and
deforming the preform at least once to form a wire product having a third cross-sectional area, the deformation step achieving a cross-sectional area reduction, wherein the third cross-sectional area is at least 30% less than the first cross-sectional area.

19. A medical device formed from the wire product of claim 18.

20. The medical device of claim 19, wherein the medical device is one of a vascular stent, a non-vascular stent, a neurovascular stent, a peripheral wire-form stent, a coil-assist stent, a septal occluder, a cable for a biostimulation device, a heart valve frame, a laser-cut tube stent for aortic grafting, or a laser-cut sheet for a hernia repair graft.
